(19) United States
(12) Patent Application Publication
(10) Pub. No.: US 2008/0274492 A1
(43) Pub. Date: Nov. 6, 2008

(54) Detection of platinum group metals with fluorophores via allylic oxidative insertion

(76) Inventors: Kazunori Koide, Pittsburgh, PA (US); Amanda L. Garner, Pittsburgh, PA (US); Fengling Song, Pittsburgh, PA (US)

Correspondence Address: MEYER UNKOVIC & SCOTT LLP, 1300 OLIVER BUILDING, PITTSBURGH, PA, 15222, US

(21) Appl. No.: 12/011,528

(22) Filed: Jan. 28, 2008

Related U.S. Application Data
(60) Provisional application No. 60/897,589, filed on Jan. 26, 2007.

(51) Int. Cl. C12Q 1/02; G01N 33/20; C07D 311/82; C07D 311/08; C07D 265/38

(52) U.S. Cl. 435/29; 436/84; 436/74; 549/388; 549/289; 544/102

(57) ABSTRACT

A method of detecting platinum group metals in a sample is provided. The method comprises the step of contacting the sample with a fluorophore capable of undergoing allylic ether or allylic ester cleavage. The fluorophore has an oxygen-protected moiety, the protecting group having an allylic functionality. A reducing agent and optionally a solubilizer are also added to the sample. Very low levels of platinum group metals such as palladium and platinum can be detected in a sample.

### FIELD OF THE INVENTION

The present invention relates to fluorogenic compounds that can be used as sensors for detection of platinum group metals such as palladium and platinum via an allylic oxidative insertion reaction.

### BACKGROUND INFORMATION

The importance of metals in biological systems and the general difficulty in measuring metals in living cells makes metal detection a particularly desirable field for the use of fluorescence technology. Some fluorescence measurement systems are useful for determining the presence of analytes in environmental samples. Finally, because certain fluorescence detection systems are rapid and reproducible, fluorescence measurements are often critical for many high-throughput screening applications.

For example, palladium is a widely used metal in chemistry, dentistry, and other materials (e.g., automotive catalytic converters, dental fillings, jewelry). In synthetic chemistry, palladium-catalyzed cross-coupling reactions in the preparation of active pharmaceutical ingredients is becoming increasingly important. However, even after purification, residual palladium is often found in the final product, which may be a health hazard. Because of such concerns, the proposed value for dietary intake is <1.5-15 μg/day per person, which is often translated into 10 ppm of palladium in active pharmaceutical ingredients as a threshold.

While typical analytical methods for detection of low levels of platinum group metals require the use of expensive spectrometers (atomic absorption spectroscopy, x-ray fluorescence, plasma emission spectroscopy), a more desirable approach would rely on detection with the naked eye.

### SUMMARY OF THE INVENTION

Accordingly, in some aspects the present invention provides compounds having the formula

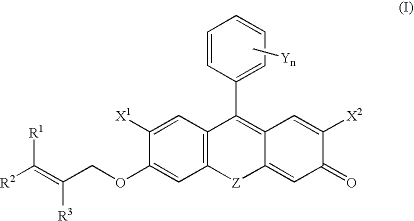

where X¹ and X² are each independently hydrogen, an alkyl group, or halogen;
Z is O, S, Se, or NR′, wherein R′ is hydrogen or an alkyl group;
n is an integer from 1 to 5;
each Y is independently hydrogen, or a functional group as that term is understood in the art; and
R¹, R², and R³ are independently either hydrogen or alkyl or aryl groups that may contain one or more further substitutions.

In another aspect, the present invention provides compounds of formula (III):

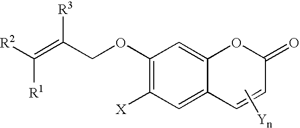

where n=1 or 2 and R¹, R², R³, X (as X¹ or X²) and Y are as defined above.

In yet another aspect, the present invention provides compounds of formula (IV):

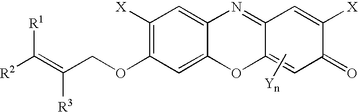

where n=1 or 2 and R¹, R², R³, X and Y are as defined above.

In additional aspects, the present invention provides a method of detecting a platinum group metal in a sample comprising the steps of 1) adjusting the pH of the sample; 2) contacting the sample with i) a fluorophore capable of undergoing allylic ether or allylic ester cleavage, ii) a reducing agent and optionally iii) a solubilizer; and 3) detecting fluorescence in the sample.

In additional aspects the present invention provides a method of detecting a platinum group metal in a sample comprising the steps of 1) adjusting the pH of the sample; 2) contacting the sample with i) a fluorophore having an O-allylic or substituted O-allylic moiety, ii) a reducing agent and optionally iii) a solubilizer; and 3) detecting fluorescence in the sample.

### BRIEF DESCRIPTION OF THE DRAWINGS

The invention is further illustrated by the following drawings in which:

FIG. 1 shows (a) palladium(0) catalyzed allyl ether cleavage; (b) fluorescent and non-fluorescent forms of fluorescein, and (c) the reaction scheme for preparation of a fluorophore of the present invention.

FIGS. 2 (a), (b) and (c) are graphs showing the relationship between amount of palladium and fluorescence. The correlation between palladium and fluorescence at 535 nm in ph 10 buffer.

(a) [1]=7.5 μM, [NaBH₄]=[morpholine]=10 mM, total volume=200 μL, 10 min incubation.

(b) [1]=7.5 μM, [NaBH₄]=[morpholine]=10 mM, total volume=100 μL, ♦=6 h incubation, ▪=12 h incubation, ▴=18 h incubation. (c) [1]=150 μM, [NaBH₄]=50 mM, [morpholine]=10 mM. total volume=200 μL, 10 min incubation.

FIGS. 3 (a), (b) and (c) are charts showing the detection of palladium in pharmaceutical samples. (a) and (b): [1]=7.5 μM, [NaBH₄]=[morpholine]=10 mM in 100 μL MeOH. In both (a) and (b), (1) blank; (2) +aspirin (1 mg); (3) +Pd (10 ng); and (4) +aspirin (1 mg) that contains Pd (10 ng), all in duplicates. (c) Left: The solution of 1 (15 μM), NaBH₄ and morpholine (both 20 mM) in MeOH from a flask not exposed to palladium reagents; Right: The same solution from a flask exposed to Pd₂ dba₃ (10 mg) in THF (3 mL) and washed.

FIG. 4 is a graph of fluorescent intensity for different concentrations of fluorophore, for Pt(II) sensing with NaBH₄.

FIG. 5 is a graph of fluorescent intensity for different concentrations of fluorophore, for Pt(IV) sensing with NaBH₄.

FIGS. 6(a)-6(d) are graphs of the relationship between pH and fluorescent intensity for different platinum species with different reducing agents.

FIGS. 7(a)-7(c) are graphs of the effect of fluorescent intensity over time for platinum (0) at varying pH.

FIGS. 8(a)-8(c) are graphs of fluorescent intensity over time for platinum (II) at varying pH.

FIGS. 9(a)-9(c) are graphs of fluorescent intensity over time for cisplatin (Pt (II) at varying pH.

FIGS. 10(a)-10(c) are graphs of fluorescent intensity over time for platinum (IV) at varying pH.

FIGS. 11(a)-11(c) are graphs of fluorescent intensity versus concentration for various platinum species.

FIG. 12(a)-12(c) are graphs of fluorescent intensity versus concentration for various platinum species after a 24 hour incubation period.

FIG. 13(a)-13(c) are graphs of fluorescent intensity over time for palladium(II) at different pH.

FIG. 14(a)-14(c) are graphs of fluorescent intensity over time for palladium(0) at different pH.

FIG. 15 (a)-(b): Photo of rock samples and solutions prepared from rock samples. Rock A, no metals; Rock B, 120 ppm Pd/Pt; Rock C, only Au/Ag; Rock D, 35 ppm Pd/Pt (Pd/Pt=3.4:1). The photo was taken above a hand-held UV lamp (365 nm).

FIG. 15(c): Naked eye detection of Pd. Vial 1: PdCl₂ (1.0 mg), dimethylglyoxime (1% w/v in ethanol) in 0.25 N HCl. Vial 2: PdCl₂ (1.0 mg), sensor, NaBH₄ in THF. Vial 3: PdCl₂ solution (30 μL, 1.0 mM), dimethylglyoxime (1% w/v in ethanol) in 0.25 N HCl. Vial 4: PdCl₂ solution (30 μL, 1.0 mM), sensor, NaBH₄ in THF.

FIG. 16 is a graph of concentration of cisplatin versus flourescence for detection of cisplatin in serum.

### DETAILED DESCRIPTION OF PREFERRED EMBODIMENTS

As used herein in the specification and claims, including as used in the examples and unless otherwise expressly specified, all numbers may be read as if prefaced by the word “about”, even if the term does not expressly appear. Also, any numerical range recited herein is intended to include all sub-ranges subsumed therein.

As used herein, the term “platinum-group metals” means elements such as platinum, palladium, ruthenium, rhodium and iridium.

The term “fluorophore” is an art-recognized term used to describe a functional group in a molecule that fluoresces. Fluorophores are well known and used extensively in biological applications such as immunochemistry. Common fluorescent labels include fluorescein and its derivatives, rhodamine and derivatives, dansyl, phycoerythrin, phycocyanin, allophycocyanin, o-phthaldehyde and fluorescamine. See, for example, Haugland, Handbook of Fluorescent Probes and Research Chemicals, Sixth Ed., Molecular Probes, Eugene, Oreg., 1996, incorporated herein by reference. Examples of additional fluorophores include cascade blue, coumarin and its derivatives, naphthalenes, pyrenes and pyridyloxazole derivatives.

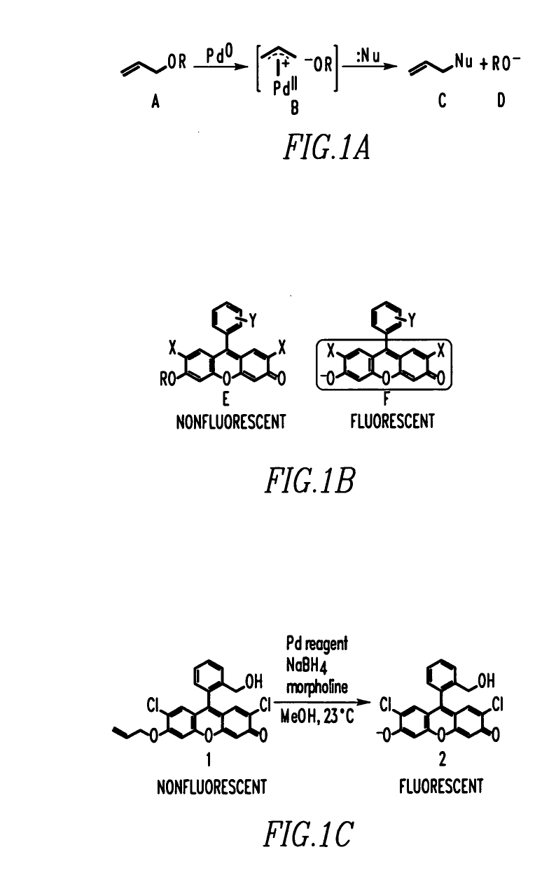

In one embodiment, palladium(0) is shown catalyzing the allylic oxidative insertion to cleave the allylic C—O bond of allylic ethers A to form palladium complexes B (FIG. 1(a)). These complexes then react with various nucleophiles to form compounds C and by-products D, which is known as the Tsuji-Trost reaction. Therefore, if A is nonfluorescent and D (or the corresponding neutral species) is fluorescent, such a system can be used to specifically detect the presence of palladium(0). Moreover, since palladium(II) can be readily reduced to palladium(0) by treating palladium(II) with reducing agents such as triarylphosphine, trialkylphosphine, and sodium borohydride, palladium(II) can also be detected by the same principle. It is expected that other platinum group metals will also catalyze the allylic oxidative insertion to cleave the allylic C—O bond of allylic ethers.

Thus, in its broadest aspect, the present invention provides a method of detecting platinum group metals in a sample comprising the steps of 1) adjusting the pH of the sample to between about pH 4 and pH 11; 2) contacting the sample with i) a fluorophore capable of undergoing allylic ether or allylic ester cleavage, ii) a reducing agent, and optionally iii) a solubilizer, and 3) detecting fluorescence in the sample. Also optionally, when the Pd or Pt in analyte (or Pd- or Pt-containing material) is not soluble in the analysis solutions, pretreatment of analytes with nitric acid or hydrochloric acid may facilitate the analysis.

In the present invention, and as would be understood by one skilled in the art, any fluorophore that is dependent upon the presence of a phenoxide or carboxylate group for fluorescence can be used to detect a platinum-group metal. Accordingly, the term “fluorophore” in the context of the present invention refers to a subset of fluorophores, those that contain a hydroxyl group to which a protecting group can be attached. The bond between the oxygen and the protecting group is cleaved in the presence of the metal, causing the fluorophore to fluoresce. Preferably, the fluorophore used in the present invention is fluorescein or derivatives of these. Also preferred are coumarin or coumarin derivatives. The fluorophore will have a substituted or unsubstituted O-allylic moiety.

“Derivatives” is an art recognized term, and refers to chemical modifications of the compounds such as substitution of halogen for hydrogen at any position in the ring or rings for multi-ring compounds, or the addition of substituents on any of the rings in the compound. Many fluorescein derivatives are known in the art; some are described, for example, in U.S. Pat. Nos. 7,160,732; 6,800,765; 7,087,766; and 5,896,094, each incorporated by reference. This list is not meant to be limiting, and is for the purpose of example only.

The fluorophore of the present invention contains a protecting group on the ring-bound oxygen, the protecting group having an allylic functionality. The term “allylic functionality” is used to mean a —H₂CCR³═CR¹R² termination on the molecule, which can be further substituted, as would be understood by one skilled in the art.

In general, hydroxyl protecting groups are well known in the art. Suitable protecting groups include, for example, alkyl, alkenyl or alkynl groups, including linear or branched embodiments of these, preferably 1-30 carbons in length, more preferably 1-20 carbons, and even more preferably, between 1 and 8 carbons; cyclic alkyl groups, such as 5 or 6 membered rings, and bicyclic or tricyclic rings; aromatic groups including aryl, alkaryl, and aralkyl groups, and groups having one or more fused rings. Any of the groups may also contain one or more heteroatoms such as a halogen, O, N, or S, and can also contain further substitutions thereon.

The term “alkyl” (or “lower alkyl”, where lower alkyl means one to six carbons) includes both “unsubstituted alkyls” and “substituted alkyls”, the latter of which refers to alkyl moieties having substituents replacing a hydrogen on one or more carbons of the hydrocarbon backbone. Such substituents may include, for example, functional groups such as a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an alkaryl, or an aromatic or heteroaromatic moiety. Such functional groups are also suitable substituents for Y in the formulas described herein (as substitutions on an aryl group), as are other functional groups known in the art and which are commonly used as substitutions on aryl groups. The term “functional group” is art recognized, and the present invention is not limited to those specifically listed.

It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls may be further substituted with alkyls, alkenyls, alkoxys, alkylthios, aminoalkyls, carbonyl-substituted alkyls, —CF₃, —CN, and the like.

In the context of the present invention, any hydroxyl protecting group with allylic functionality can be used, so that the bond between the terminal atom of the protecting group and the oxygen on the fluorophore is susceptible to cleavage by the metal. One skilled in the art can easily determine if a particular fluorophore with a protecting group can work in the method of the present invention, simply by observing the compound on exposure to the metal. If a change in color or fluorescence is detected, the compound is suitable for use as a metal detector. Preferably, the protecting group is an allyl or substituted allyl group. Preferably, the fluorophore selected is one that emits light in the ultraviolet or visible spectrum upon contact with the metal. To detect the presence of a metal in a sample, the sample is contacted with the fluorophore of the present invention. If the sample fluoresces (as determined with a UV lamp, laser pen, or other device or by visual observation), then the presence of the metal is confirmed. Visual detection is possible, using the sensors and methods of the present invention, at part-per-million (ppm) levels.

The sample containing the platinum group metal of interest (to be detected) is adjusted to a pH between about 4 and 11 and then contacted with the fluorophore, a reducing agent, and optionally a solubilizer.

The amount of fluorophore added to the sample will vary somewhat, based on the level of detection desired and the size of the sample. Preferably, the fluorophore will be added in concentrations ranging from about 1 micromolar (μM) to about 250 μM, based on a 50 μL to 5 mL sample size. More preferably, the concentration of fluorophore added is between about 2 μM to about 200 μM, most preferably between about 5 μM and about 20 μM per any volume of the final solution determined by instrument (e.g., 100-200 μL if a plate reader is used, 0.5-3 mL if a fluorometer is used).

A reducing agent is also added, such as hydrides including NaBH₄ or NaBH(OAc)₃, and triaryl- or trialkylphosphines such as PPh₃ or other strongly electron donating phosphines. The concentration of reducing agent added is between about 200-300 micromolar in the case of phosphines, for example, and between about 7-33 millimolar for hydrides, per any volume of the final solution.

A buffer (for pH adjustment) and optionally a solubilizer are also added to the sample. Suitable solubilizers include, for example, dimethyl sulfoxide (DMSO), tetrahydrofuran (THF) and dimethyl formamide (DMF). Non-volatile solubilizers are preferred. Commercially available buffers at the desired pH are well known in the art. The amount of solubilizer used will be about 5-20 volume %, and the amount of buffer will be about 80-95%, volume % based on the combined volume of buffer and solubilizer.

In some preferred embodiments, the present invention provides compounds of the formula (I):

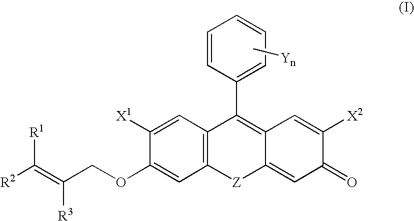

where X¹ and X² are each independently hydrogen, an alkyl group, or halogen;
Z is O, S, Se, or NR′, wherein R′ is a hydrogen or an alkyl group.
n is an integer from 1 to 5;
each Y is independently hydrogen, or a functional group as that term is understood in the art; and
R¹, R², and R³ are independently either hydrogen, alkyl, or aryl groups that may contain one or more further substitutions.

In one embodiment n=1 and Y is OH. In another embodiment n=1, Y is OH and at least one of X¹ and X² is CL. In yet another embodiment, at least one of R¹, R², R³ are hydrogen, n=1, Y is OH and at least one of X¹ and X² is CL.

In a preferred embodiment, the fluorophore (with the protecting group) is a compound having the formula (II)

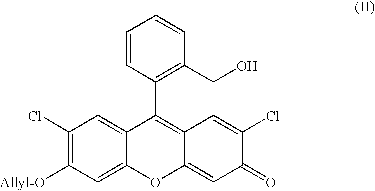

In additional embodiments, the fluorophore is a compound having formula (III):

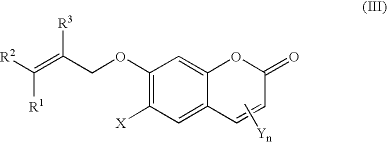

where R¹, R², R³, X, and Y are as defined above, and n=1 or 2. In one embodiment n=1 and Y is OH. In another embodiment n=1, Y is OH and at least one X is CL. In yet another embodiment, at least one of R¹, R², R³ is hydrogen, n=1, Y is OH and X is CL.

In other embodiments, compounds of formula (IV) are provided:

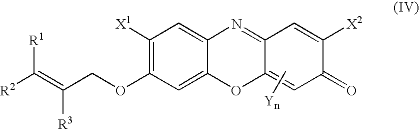

where n=1 or 2 and R¹, R², R³, and Y are as defined above, each X is independently hydrogen, alkyl group, or halogen. In one embodiment n=1 and Y is OH. In another embodiment n=1, Y is OH and at least one X is CL. In yet another embodiment, at least one of R¹, R², R³ is hydrogen, n=1, Y is OH and at least one X is CL.

The methods of the present invention are suitable for detection of platinum group metals having different oxidation states. Preferably, the metal of the present invention is platinum or palladium. For example, palladium(II) and palladium(0) can be detected with the present methods. Platinum (0), platinum (II) and platinum (IV) can be detected with the present methods. Amounts as low as about 0.5 parts per billion (ppb) in a 5 μg sample can be detected with the methods of the present invention.

Accordingly, the present invention provides a platinum group metal sensor that relies on observation of the fluorescence emission with a simple hand-held long-range UV lamp or laser pen. The methods of the present invention can be used in numerous scenarios, such as detection of platinum-group metals in pharmaceutical samples; biological samples, including cells, blood, plasma, serum, saliva, urine, tears, sweat, cerebrospinal fluid or other tissues; in environmental samples, such as water, air, wastewater, soil or sludge; drinking water; water used for preparing compositions for human contact or consumption, such as cosmetic preparations and food supplements; mining site samples and mining-related materials such as ore, mining waste and the like. Samples can be gases, liquids or solids.

The methods of the present invention can also be used to detect platinum-group metals in samples prepared from a solution or extract of a pharmaceutical preparation, extracts from vessels used for carrying out chemical and biological reactions, and solutions or extracts from other sources, such as metallic and non-metallic solids.

### EXAMPLES

The following examples are intended to illustrate the invention and should not be construed as limiting the invention in any way.

### Example 1

### Preparation of Oxygen-Protected Fluorophore (Compound 1)

Fluorescein compounds are non-fluorescent when the phenolic hydroxy group is alkylated (FIG. 1 (b), E) while strongly green fluorescent (φ˜0.9) when the hydroxy group is deprotonated (FIG. 1 (b). This principle has been used for various purposes, primarily in biology for fluorescent imaging. The same chemical principle can be used for palladium sensing in a scenario where compounds A and D correspond to E and F, respectively. As compound 1, the allyl ether 1 (FIG. 1 (c) was prepared in two steps from commercially available 2′,7′-dichlorofluorescein in multiple gram quantities.

To a solution of bis-O-allyl 2′,7′-dichlorofluorescein (5.62 g, 11.7 mmol) in CH₂Cl₂ at −78° C. under a nitrogen atmosphere was added diisobutylaluminum hydride (about 1.0 M in hexanes, 42.1 mL, 42.1 mmol). The reaction mixture was warmed to about 0° C. for about 1.5 h, and then quenched with saturated NH₄Cl (12.6 mL). Et₂O (58.5 mL) was added to the solution at about 0° C. followed by 2,3-dichloro-5,6-dicyano-1,4-benzoquinone (about 2.00 g, 8.81 mmol). The resulting solution was allowed to stir for about 15 min at 0° C. The mixture was filtered through a pad of Celite, eluting with Et₂O and concentrated in vacuo. The crude residue was purified by recrystallization from EtOAc and hexanes to afford the allyl intermediate as an orange solid (about 4.4 g, 88%).

With this compound in hand, qualitative experiments were performed, showing that the treatment of compound 1 with catalytic (0.5-2 mol %) Pd(PPh₃)₄, excess NaBH₄ and excess morpholine in THF/MeOH instantaneously generated the red material 2 at about 23° C. nearly quantitatively. The same conversion was achieved when Pd(OAc)₂ was used in lieu of Pd(PPh₃)₄. Because both of the palladium oxidation states (0 and +2) could be present in fine chemicals and pharmaceutical ingredients after palladium-catalyzed cross-couplings, these successful transformations of 1 to 2 indicate that detection of palladium species with the naked eye is possible.

### Example 2

### Quantitative Analysis

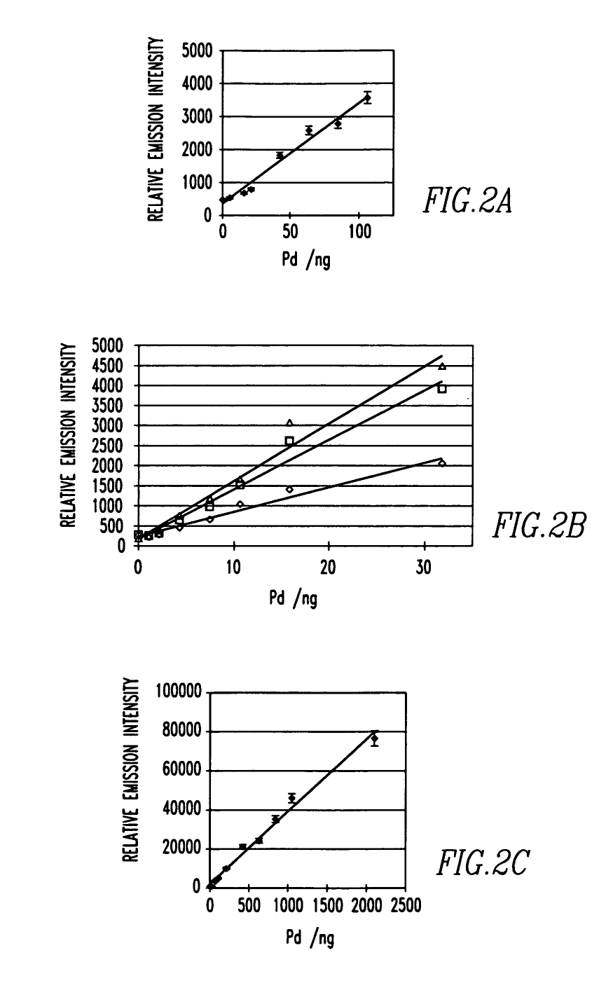

In the pharmaceutical industry, 5 mg samples are routinely used for palladium analyses. The final solutions in each well of a polypropylene 96-well plate consisted of Pd(PPh₃)₄ in various quantities and compound 1 at 7.5 μM in 200 μL of MeOH. Fluorescent signals could be easily detected after less than 10 min incubation at about 23° C. and were linearly correlated to the palladium quantities from 20 ng to 100 ng (FIG. 2a). In order to detect less palladium, the reaction solutions were incubated longer (about 6-18 h) to increase the sensitivity of the method to <5 ng palladium (FIG. 2b). By increasing the concentration of 1 from 7.5 to 150 μM, the linear range could be extended to 2 μg of palladium (FIG. 2c). This result, together with the determination of the correlation between the concentrations of Compound 2 and its fluorescent signals, the turnover number (TON) in this system was determined to be approximately 700.

### Example 3

### Detection of Palladium in Pharmaceutical Samples

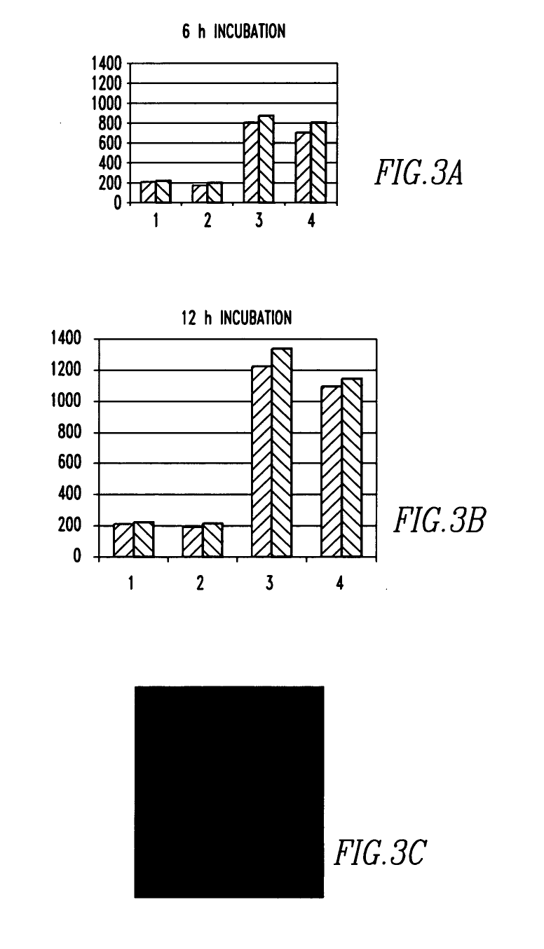

To determine whether this fluorescent sensor could be applied to the palladium analysis in pharmaceutical products, a sample was prepared containing a commercially available aspirin tablet and Pd(PPh₃)₄ at the 10 ppm level. A solution (100 μL) of this sample containing 1 mg of the drug and 10 ng of atomic palladium was added to the MeOH solution of 1, NaBH₄, and morpholine (final concentrations: about 7.5 μM, 10 mM, 10 mM, respectively). The fluorescent intensity of this solution (FIG. 3, column 4) was then measured and compared to a positive control (aspirin-free palladium solution; column 3) and a negative control (palladium-free aspirin solution and blank; columns 1 and 2, respectively); the signals from the palladium-contaminated aspirin solutions were nearly the same as the positive controls, supporting the robustness of the palladium sensing method under very heterogeneous conditions.

### Example 4

### Quality Control

An additional concern about palladium contamination is the reactors used for palladium-catalyzed reactions. To test whether the method can detect residual palladium in a reactor, a THF solution of Pd₂(dba)₃ (10 mg) was stirred in a 10 mL round-bottom flask for about 1 h at about 23° C. After standard laboratory washing procedure with water and acetone, the palladium sensor solution ([1]=15 μM, NaBH₄] and morpholine each 20 mM in MeOH) was added to this flask and stirred for 1 h at 23° C. Presumably due to residual palladium on the glass surface, the solution became more green fluorescent, showing that this palladium sensing method can be used for quality control.

### Example 5

### Reducing Agents

To a disposable 4.5-mL cuvette was added 20% DMSO/80% pH 10 buffer (4.0 mL), Pt (625-2.5 nM final concentrations, in various oxidation states), reducing agent (see below for exact compounds tested), and sensor (5 μL of 10 mM solution in DMSO). The cuvettes were incubated at 24° C. for 20-24 h before fluorescence measurement. Many reducing agents were tested based on to commercial availability and known ability to participate in Pd- and Pt-catalyzed reactions. Solvent used was 20% DMSO/80% pH 10 buffer.

### Reducing Agents:

Unsuccessful (non-phosphine): ammonium formate (NH₄HCO₂) (5 mg), triethylsilane (Et₃SiH) (10 μL of 100 mM DMSO solution).

Moderately successful (non-phosphine): sodium triacetoxyborohydride (NaBH(OAc)₃) (300 μL of 100 mM solution in pH 7 buffer).

Moderately successful (phosphine): tri-2-furyl phosphine, tri-o-tolylphosphine, 2-(di-^{t}butylphosphino)biphenyl, 1,1′-bis(diphenylphosphino)ferrocene, tricylclohexylphosphine, bis(2-diphenylphosphinophenyl)ether (10 μL of 100 mM solution in DMSO was used for each phosphine/phosphite).

The combination of NaBH₄ and phosphine was also moderately successful (7.5-33 mM NaBH₄+250 mM phosphine).

Since the solvent was expected to only have a minor effect (as shown in the kinetic data), it was assumed that all of these conditions would give the same negative result in 20% DMSO/80% pH 7 buffer.

### Example 6

### Pt(II) Sensing with NaBH₄

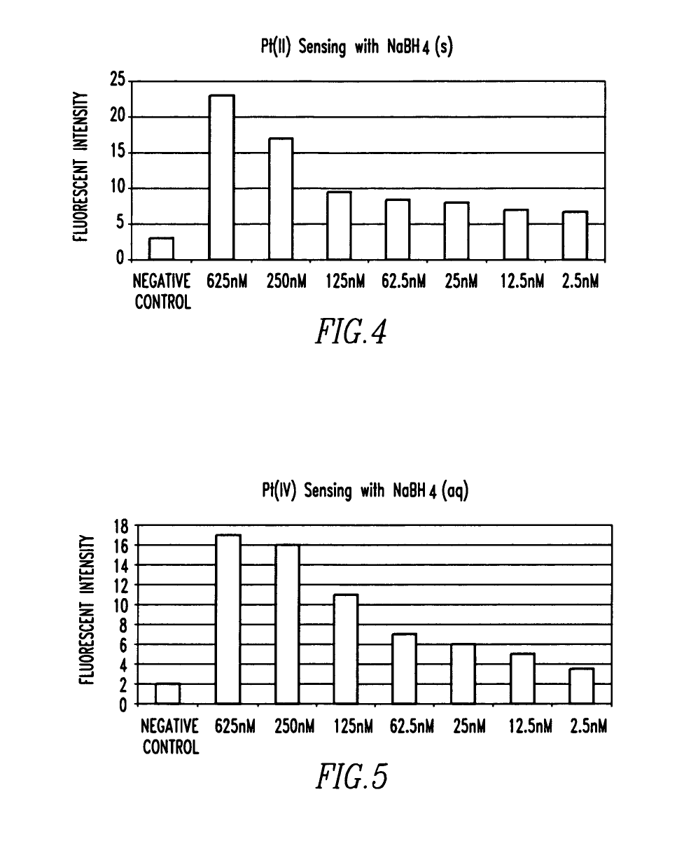

To a disposable cuvette was added 4.0 mL of 20% DMSO/80% pH 7 buffer followed by varying amounts of Pt (625-2.5 nM final concentrations; see Pt species listed above). To the mixture was then added NaBH₄ (5.0 mg) and sensor (5.0 μL of 10 mM solution in DMSO). The samples were shaken to mix and allowed to incubate for 1 h at 24° C. before fluorescence measurement. Detection to 2.5 nM was consistently observed over triplicate experiments. Results are shown in FIG. 4.

### Example 7

### Pt(IV) Sensing with NaBH₄

To a disposable cuvette was added 20% DMSO/80% pH 10 buffer (4.0 mL) followed by varying amounts of Pt (625-2.5 nM final concentrations; see Pt species listed above). To the mixture was then added NaBH₄ (300 μL of 100 mM solution in pH 7 buffer) and sensor (5.0 μL of 10 mM solution in DMSO). The samples were shaken to mix and allowed to incubate overnight (20-24 h) at 24° C. before fluorescence measurement. Detection to 2.5 nM was consistently observed over triplicate experiments. Results are shown in FIG. 5.

### Example 8

### pH Studies

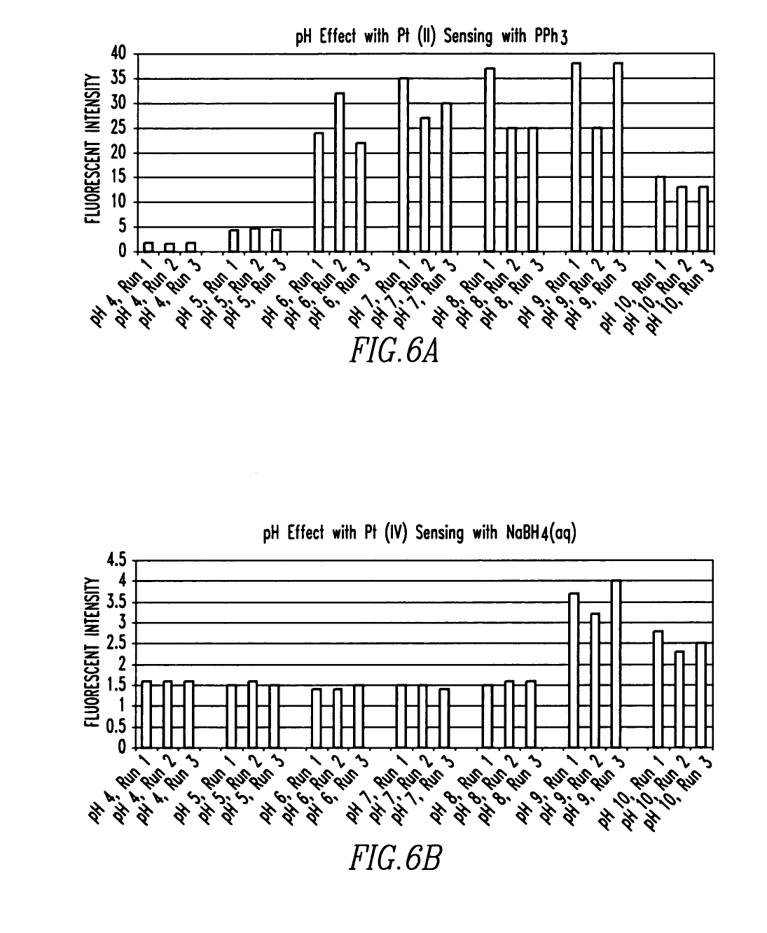

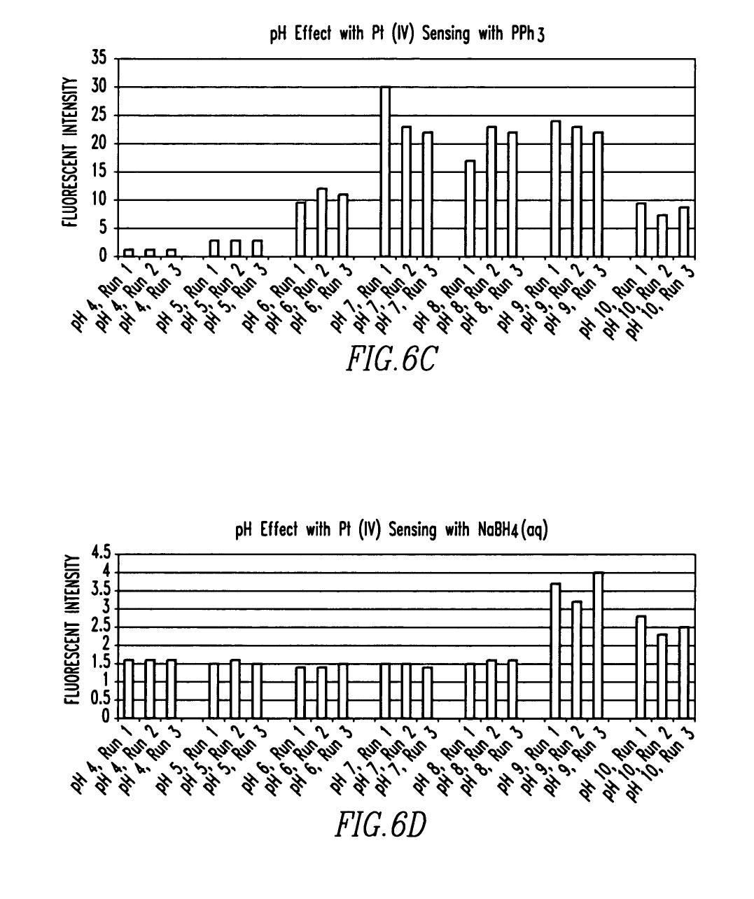

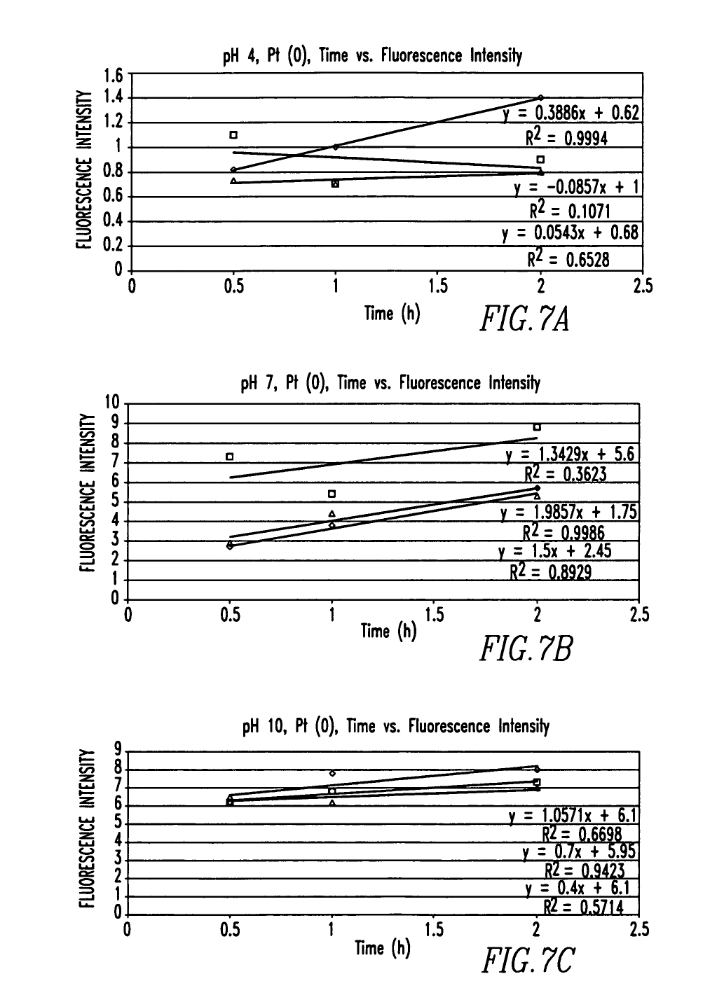

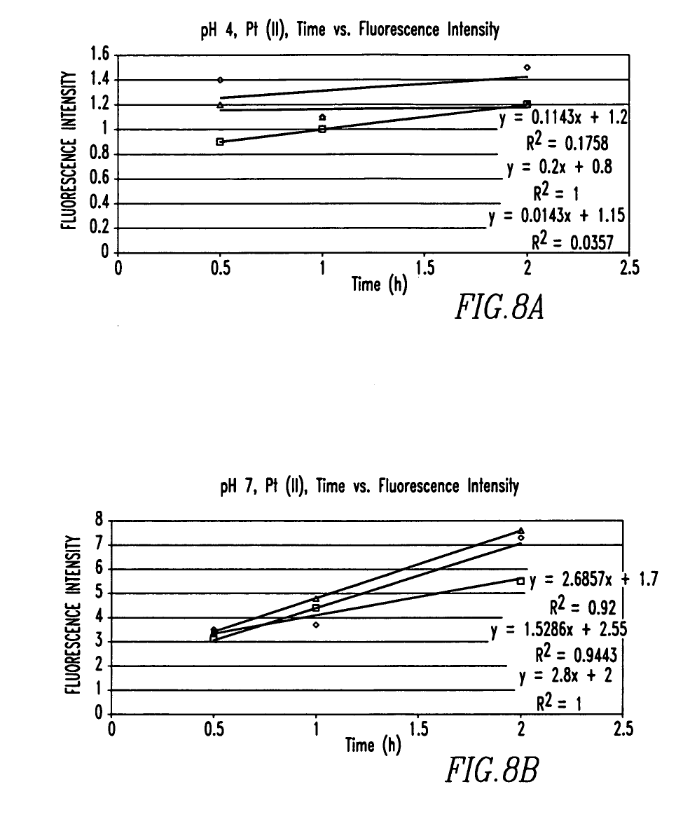

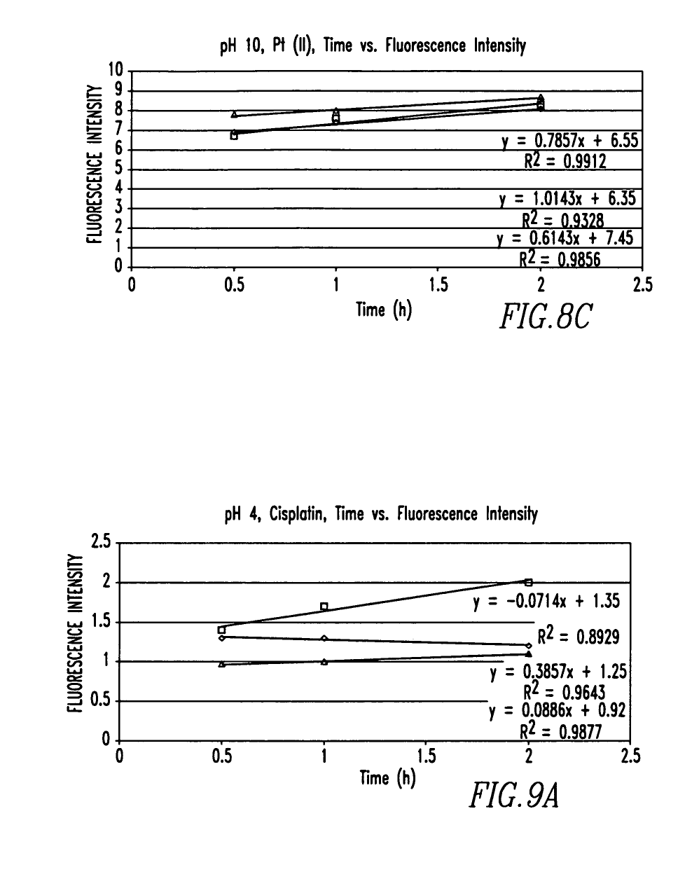

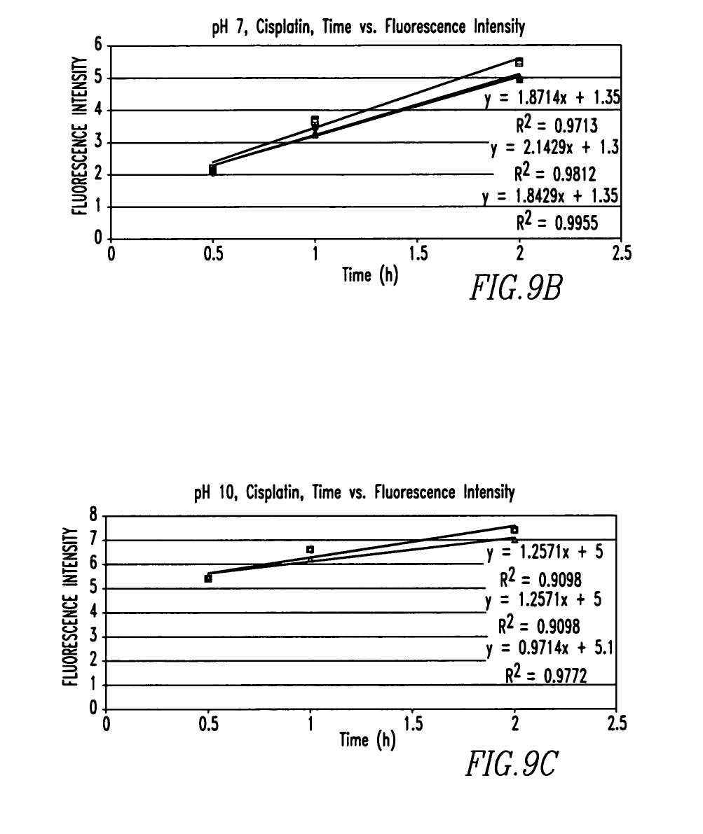

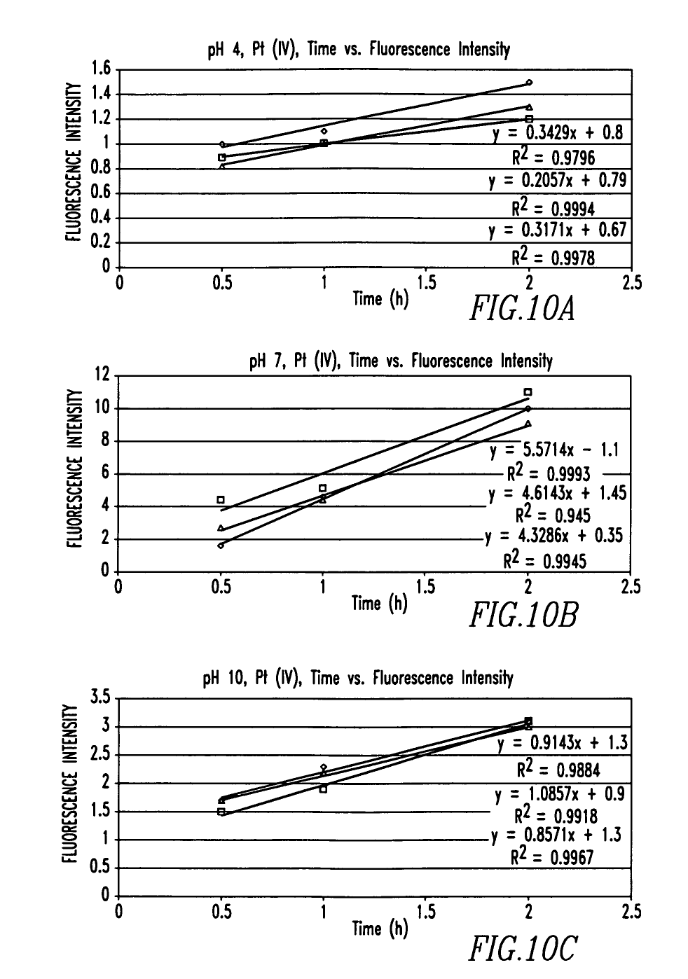

To a disposable cuvette was added 20% DMSO/80% pH xx buffer (4.0 mL; xx=4-10) followed by varying amounts of Pt (625-250 μM final concentrations; Pt species=PtCl₂ or H₂PtCl₆). To the mixture was then added reducing agent (PPh₃: 10 μL of 100 mM solution in DMSO; or NaBH₄: 300 μL of 100 mM solution in pH 7 buffer) and sensor (5.0 μL of 10 mM solution in DMSO). The samples were shaken to mix and allowed to incubate for 1 h at 24° C. before fluorescence measurement. The results for each are shown in FIGS. 6a, 6b and 6c:

(i) PtCl₂ with PPh₃: reaction does proceed at pH 6-10 although to a lesser extent at pH 10. pH 7 and pH 9 showed the highest average intensity. These results together with the kinetic data led to the use of 20% DMSO/80% pH 7 buffer. Also, pH 7 buffer is more commonly used than pH 9.

(ii) PtCl₂ with NaBH₄: reaction does not proceed from pH 4-8. The reaction is successful at pH 9, 10; which is most likely due to the stability of the reducing agent.

(iii) H₂PtCl₆ with PPh₃: reaction does not proceed at pH 4, 5; the reaction is moderately successful at pH 6, and 10; and the reaction is most successful from pH 7-9. These results together with the kinetic data led to the use of 20% DMSO/80% pH 7 buffer. Also, pH 7 buffer is more commonly used than pH 8, 9.

(iv) H₂PtCl₆ with NaBH₄: reaction does not proceed from pH 4-8. The reaction is successful at pH 9, 10; which is most likely due to the stability of the reducing agent.

### Example 9

### Platinum Kinetic Studies

To a stirring solution of 50:50 DMSO/buffer (buffer=pH 4, 7, 10) was added Pt (50 μL of 5 mM Pt solution; Pt(0)=Pt(PPh₃)₄ in DMSO, Pt(II)=PtCl₂ in DMSO, Pt(II)=trans-Pt(NH₃)₂Cl₂ in 1% HNO₃, Pt(IV)=H₂PtCl₆ in 1% HNO₃), PPh₃ (200 μL of 100 mM solution in DMSO), and sensor (10 mg). Fluorescence measurements were taken at 30 min, 1 h, and 2 h to determine the effect of pH on the kinetics of the reaction. Samples for fluorescence measurement were prepared in the following manner: 100 μL of the reaction mixture at each time designated was diluted with 4 mL of 20% DMSO/80% pH 7 buffer. Each experiment was run in triplicate.

### Platinum Species

Pt(PPh₃)₄ was chosen as an example of Pt(0) because it is analogous to the commonly used Pd(PPh₃)₄, it is commercially available, and soluble in DMSO. PtCl₂ was chosen as an example of Pt(II) because it contains dissociable chloride ligands, it is commercially available, and soluble in DMSO. trans-Pt(NH₃)₂Cl₂, or Cisplatin, was chosen because it is an example of a platinum-based cancer drug. Cisplatin also contains non-dissociable amine ligands from which we can deduce the effect of ligands on the platinum (II) species by comparing the results from Cisplatin and PtCl₂. H₂PtCl₆ was chosen as an example of Pt(IV) because it is readily soluble in aqueous acid.

The studies are generalizable to all platinum species as indicated by the kinetic study. All exhibit similar kinetics at pH 4, 7, and 10 with pH 7 showing the fastest rate. This leads to two conclusions: (1) All oxidation states (0, II, IV) react in a similar manner; and (2) The ligands on Pt have no effect on the reaction.

### Platinum Sensing Using PPh₃ as a Reducing Agent

### (A) pH Dependence Kinetic Studies

In order to determine the effect of pH on the platinum sensing reaction, a kinetic study at pH 4, 7, and 10 was undertaken, examining the most common oxidation states (0, II, IV) of the metal. The results are shown in FIGS. 7a-d, 8a-c, 9a-c and 10a-c for each pH and platinum species. For all oxidation states, the rate was fastest at pH 7, followed by pH 10, and essentially no reaction at pH 4.

### (B) Detection to Low nM Concentrations of Platinum

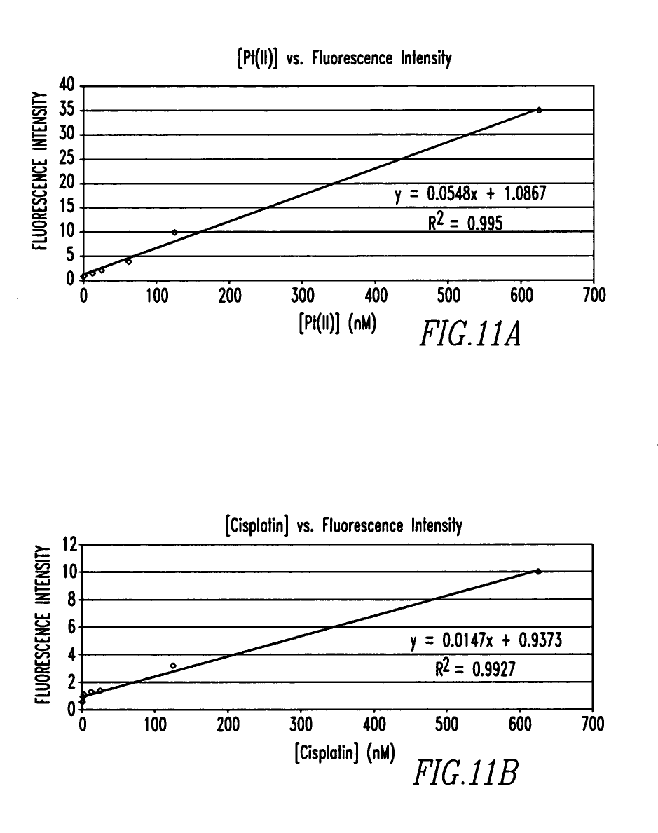

All further sensing experiments used 20% DMSO/80% pH 7 buffer instead of 20% DMSO/80% pH 10 buffer due to the results of the pH kinetic study. Using the previously outlined method, platinum sensing (Pt(II), Pt(IV), and Cisplatin) to low nM concentration (625 nM to 1.25 nM) was performed. The reactions were run at 24° C. for 20-24 h. The results for each platinum species are shown in FIGS. 11a-c.

### (C) Detection to pM Concentrations of Platinum

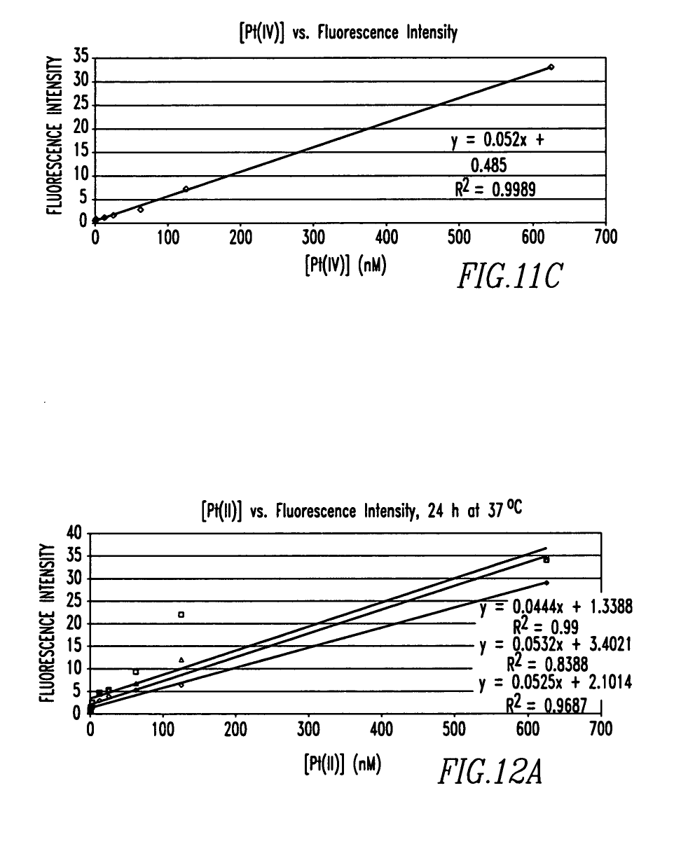

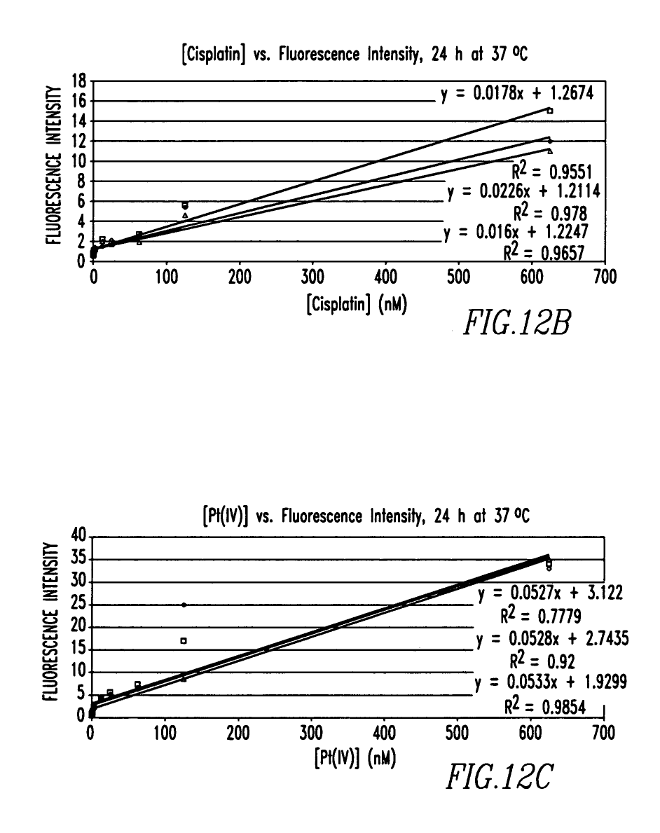

Using the previously outlined method, platinum sensing (Pt(II), Pt(IV), and Cisplatin) to pM concentration (625 nM to 250 pM) was carried out. The reactions were run at 37° C. for 20-24 h. The results for each platinum species are shown in FIGS. 12a-c.

### Example 10

### Palladium Kinetic Studies

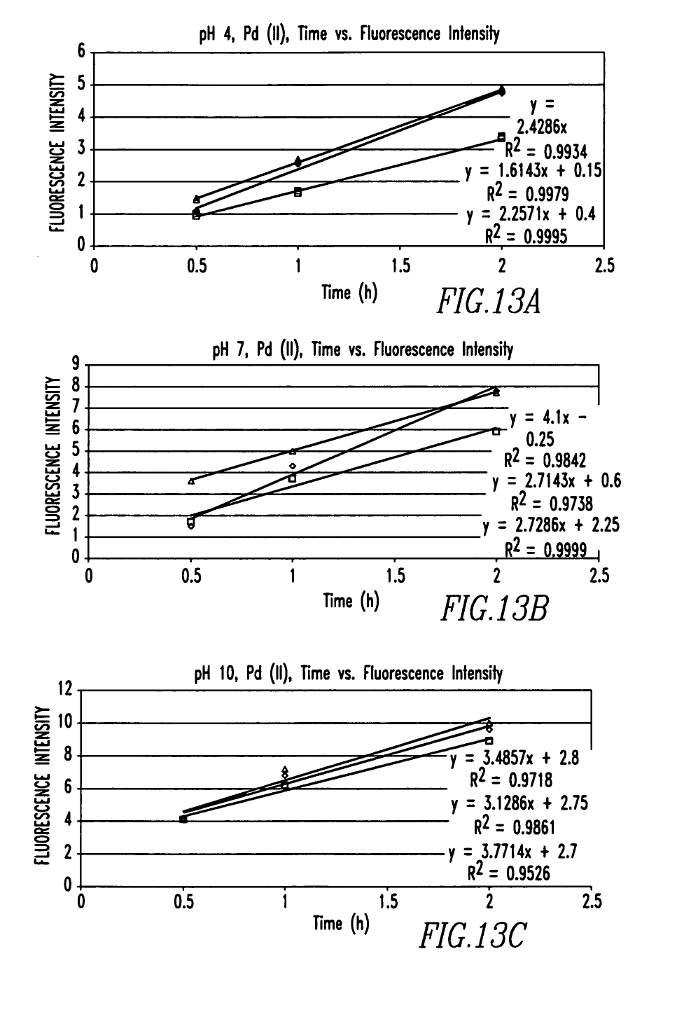

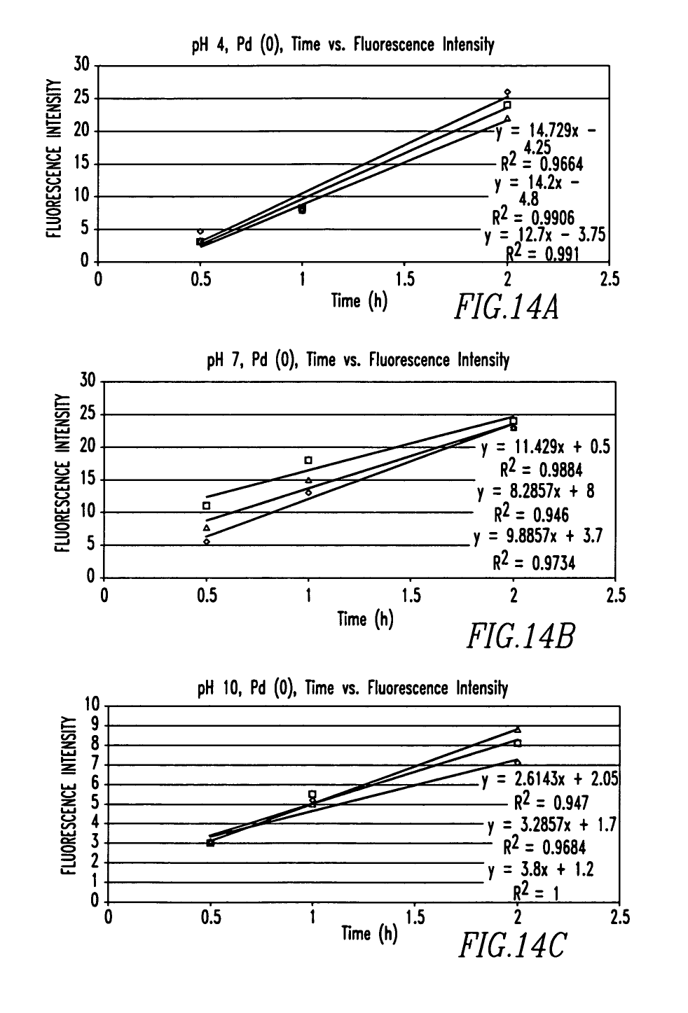

To a stirring solution of 50:50 DMSO/buffer (buffer=pH 4, 7, 10) was added Pd (50 μL of 5 mM Pd solution; Pd(0)=Pd(PPh₃)₄ in DMSO, Pd(II)=PdCl₂ in DMSO), PPh₃ (200 μL of 100 mM solution in DMSO), and sensor (10 mg). Fluorescence measurements were taken at 30 min, 1 h, and 2 h to determine the effect of pH on the kinetics of the reaction. Samples for fluorescence measurement were prepared in the following manner: 100 μL of the reaction mixture at each time designated was diluted with 4 mL of 20% DMSO/80% pH 7 buffer. Each experiment was run in triplicate. The results are shown in FIGS. 13a-c and 14a-c. The results show that the reaction with Pd(II) is fastest at pH 10 followed by 7. For Pd(0), the reaction is fastest at pH 7 followed by 10.

### Example 11

### Ore

Current methods of discovering Pd/Pt-containing rocks (Pd and Pt coexist in most rocks) involve atomic absorption analysis, which miners cannot employ at the mining site. As an additional application of the sensor technology, rock samples from mining were obtained from a mining company.

Rock stock solutions (50 μL each) were prepared as follows: 5 g of rock species was fused for 1 h at 650° C. in an oven. After being cooled to room temperature, the melt was boiled in 75 mL of concentrated HCl for 15 min. After being cooled, 25 mL of concentrated HNO₃ was added and soaked over night. The resulting mixture was boiled for 2 h to evaporate to near dryness. After drying, 25 mL of aqua regia was added and evaporated to a wet salt, this mixture was dissolved and diluted with 100 mL of water, filterer, and the filtrate was kept for use as rock stock solution. 50 μL of the rock stock solution was added to sensor solution (2, 10 μM; PPh₃, 100 μM in pH 10.0 borate buffer (5 mL) to obtain the rock samples.

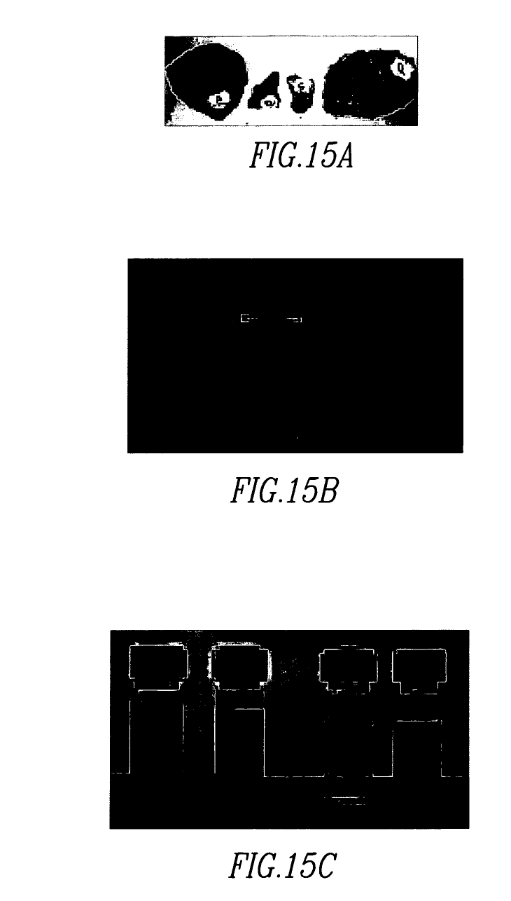

The rock stock solutions were mixed with Pd sensor solution for 1 h at 24° C. and the fluorescent intensity of the resulting solution was then measured. The rocks are shown in FIGS. 15a and 15b (FIG. 15b is prepared samples).

Rock B contains Pd/Pt (3.4:1, 120 ppm), an economically viable quantity. Rock D contains 30% of Pd/Pt compared to rock B. Rock A contains no transition metals and rock C contains Au/Ag but no Pd/Pt. Only rocks containing Pd/Pt (samples B, D) converted 2 to 3 and the fluorescent intensity was relative to the amount of Pd/Pt in the sample. Rock samples A and C were negative controls and exhibited negligible fluorescence demonstrating the viability of our sensor in Pd/Pt detection at mining sites via a simple hand-held UV lamp.

For a sensor to be applicable in high-throughput screening, it is desirable to rely on detection with the naked eye. To demonstrate that Pd can be detected even without a UV lamp, four solution, two containing the Pd sensor (vials 2 and 4) and two containing PdCl₂ and no Pd sensor as a negative control (vials 1 and 3) were prepared. Dimethylglyoxime was added to the negative controls to coordinate to Pd. As FIG. 15c indicates, only those solutions containing the sensor exhibited a color change detectable with the naked eye showing the usefulness of the sensor.

### Example 12

### Cisplatin Detection in Human Serum

While the dosage of Cisplatin is currently based on body surface area, there are many disadvantages associated with this method. Insufficient platinum drug is ineffective and excess platinum drug presents a number of toxicities including nephrotoxicity, myelosuppression, ototoxicity, anaphylaxis, and peripheral neuropathies. After injection of Cisplatin into the bloodstream, the drug is quickly bound by proteins while the free fraction of the drug is removed through excretion via the kidneys. Because this drug negatively affects the kidneys, patients may or may not retain efficient mechanisms for excess drug removal. As a result, sensitive detection methods are necessary for determining the biodistribution of cisplatin, namely in whole blood, plasma, or serum. Using the sensing method of the present invention, we have shown detection of cisplatin to 100 nM in human serum samples can be accomplished. (This is about as sensitive as previous studies with ICP-MS and MS (512 nM, 256 nM, respectively).

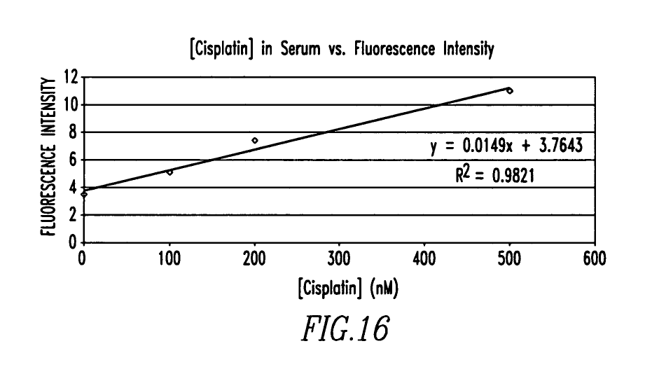

To 50 μL of human serum in a disposable cuvette was added pH 7 buffer followed by a solution of cisplatin in pH 7 buffer (final concentrations of cisplatin in serum=500=100 nM). The total volume of pH 7 buffer added from both solutions was 100 μL (2-fold dilution of serum). To this mixture was then added PPh₃ (10 μL, 100 mM in DMSO) and sensor (5 μL, 10 mM in DMSO). The cuvettes were incubated at 24° C. for 20-24 h before fluorescence measurement. The results are shown in FIG. 16.

Whereas particular embodiments of this invention have been described above for purposes of illustration, it will be evident to those skilled in the art that numerous variations of the details of the present invention may be made without departing from the invention as defined in the appended claims.

What is claimed is:

1. A method of detecting a platinum group metal in a sample comprising the steps of 1) adjusting the pH of the sample; 2) contacting the sample with i) a fluorophore capable of undergoing allylic ether or allylic ester cleavage, ii) a reducing agent and optionally iii) a solubilizer; and 3) detecting fluorescence in the sample.

2. A method of detecting a platinum group metal in a sample comprising the steps of 1) adjusting the pH of the sample; 2) contacting the sample with i) a fluorophore having an O-allylic or substituted O-allylic moiety, ii) a reducing agent and optionally iii) a solubilizer; and 3) detecting fluorescence in the sample

3. The method of claim 1, wherein the platinum group metal is palladium.

4. The method of claim 1, wherein the platinum-group metal is platinum.

5. The method of claim 1, wherein the fluorophore is fluorescein or a derivative thereof.

6. The method of claim 1, wherein the fluorophore is coumarin or a derivative thereof.

7. The method of claim 1, wherein the protecting group is a substituted or unsubstituted C₁-C₈ alkenyl group.

8. The method of claim 7, wherein the protecting group is a substituted or unsubstituted allyl group.

9. The method of claim 1, wherein the sample is a biological sample selected from the group consisting of cells, whole blood, plasma, serum, saliva, urine, sweat, tears, cerebrospinal fluid and solid tissue.

10. The method of claim 1, wherein the sample is an environmental, chemical or mining sample selected from the group consisting of soil, water, ore, air and waste.

11. The method of claim 1, wherein the sample is selected from the group consisting of a solution or extract of a pharmaceutical preparation, a solution or extract of a biological specimen, an extract from a vessel used for carrying out chemical or biological reactions, and a solution or extract from a metallic or non-metallic solid.

12. The method of claim 1, wherein the reducing agent is NaBH₄ or phosphine (PPh₃).

13. The method of claim 1, wherein the fluorophore has the formula 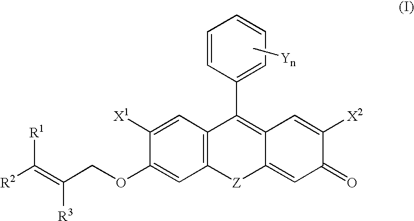
where X¹ and X² are each independently hydrogen, alkyl group, or halogen;
Z is O, S, Se, or NR′, wherein R′ is a hydrogen or an alkyl group;
n is an integer from 1 to 5;
each Y is independently hydrogen, or a functional group; and
R¹, R², and R³ are independently either hydrogen atom, alkyl, or aryl groups that may contain one or more further substitutions.

14. The method of claim 13, wherein the compound has the formula

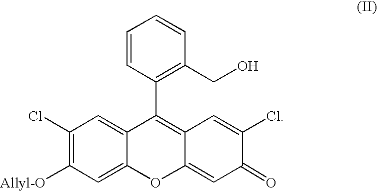

15. The method of claim 13, wherein Y is OH.

16. The method of claim 15, wherein at least one of X¹ and X² is Cl.

17. The method of claim 16, wherein at least one of R¹, R², and R³ is hydrogen.

18. A compound having the formula 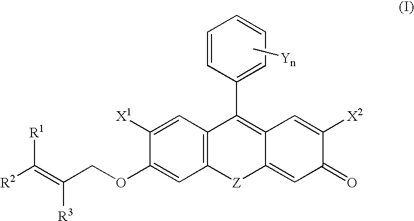
where X¹ and X² are each independently hydrogen, alkyl group, or halogen;
Z is O, S, Se, or NR′, wherein R′ is an alkyl group.
n is an integer from 1 to 5;
each Y is independently hydrogen, or a functional group; and
R¹, R², and R³ are independently either hydrogen atom, alkyl, or aryl groups that may contain one or more further substitutions.

19. The compound of claim 18, wherein Y is OH.

20. The compound of claim 19, wherein at least one of X¹ and X² is Cl.

21. The compound of claim 20, wherein at least one of R¹, R², and R³ is hydrogen.

22. A compound having the formula

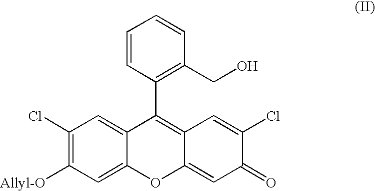

23. A compound having the formula 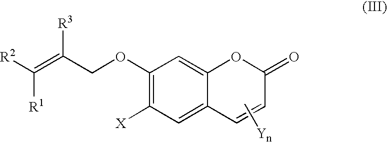
where X is hydrogen, alkyl group, or halogen;
n is an integer from 1 to 2;
each Y is independently hydrogen, or a functional group; and
R¹, R², and R³ are independently either hydrogen atom, alkyl, or aryl groups that may contain one or more further substitutions.

24. The compound of claim 23, wherein Y is OH.

25. The compound of claim 24, wherein at least one of X¹ or X² is Cl.

26. The compound of claim 25, wherein at least one of R¹, R², and R³ is hydrogen.

27. A compound having the formula: 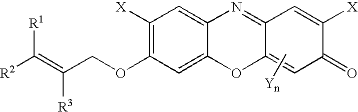
where each X is independently hydrogen, alkyl group, or halogen;
n is an integer from 1 to 2;
each Y is independently hydrogen, or a functional group; and
R¹, R², and R³ are independently either hydrogen atom, alkyl, or aryl groups that may contain one or more further substitutions.

28. The compound of claim 27, wherein Y is OH.

29. The compound of claim 28, wherein at least one of X¹ or X² is Cl.

30. The compound of claim 29, wherein at least one of R¹, R², and R³ is hydrogen.
